(19) United States
(12) Patent Application Publication
(10) Pub. No.: US 2024/0225618 A1
(43) Pub. Date: Jul. 11, 2024

(54) DEVICE FOR COLLECTING A FRACTION OF THE PERSPIRATION EXCRETED BY A SUBJECT

(71) Applicants: UNIVERSITE DE LILLE, LILLE (FR); CENTRE HOSPITALIER UNIVERSITAIRE DE LILLE, LILLE Cedex (FR); BE-LAB, WAMBRECHIES (FR)

(72) Inventors: Jean-Philippe GARNIER, LILLE (FR); Régis LOGIER, MARQUETTE LEZ LILLE (FR); Jean-Claude DARCHEVILLE, Le Touquet Paris-Plage (FR)

(21) Appl. No.: 18/559,183

(22) PCT Filed: Apr. 14, 2022
(86) PCT No.: PCT/FR2022/050709

§ 371 Date: Nov. 6, 2023

(30) Foreign Application Priority Data
May 6, 2021 (FR) FR2104777

(51) Int. Cl. A61B 10/00; A61B 5/00; A61B 5/145; A61B 5/1477; A61F 13/534; A61F 13/537; A61F 13/84

(52) U.S. Cl. CPC A61B 10/0064; A61B 5/002; A61B 5/14517; A61B 5/14539; A61B 5/1477; A61B 5/4266; A61B 5/6833; A61F 13/53713; A61B 2010/0006; A61B 2562/08; A61F 2013/53445; A61F 2013/53721; A61F 2013/8473

(57) ABSTRACT

A device for collecting a fraction of the perspiration excreted by a subject, the device being of the type including a multi-layer patch including: —a first layer including at least one perspiration-collecting aperture and a transfer opening; —a hydrophilic absorbent layer, positioned above the first layer and communicating with the external environment, and communicating with the first layer via the transfer opening; —a film, possibly transparent, that constitutes a barrier against water and against vapour, and that covers at least the upper face of the absorbent layer; the device notably including means for regulating the rate at which perspiration in the liquid and/or vapour state penetrates into the absorbent layer.

### CROSS REFERENCE TO RELATED APPLICATIONS

This application is a National Stage of International Application No. PCT/FR2022/050709, having an International Filing Date of 14 Apr. 2022, which designated the United States of America, and which International Application was published under PCT Article 21(2) as WO Publication No. 2022/234204 A1, which claims priority from and the benefit of French Patent Application No. 2104777 filed on 6 May 2021, the disclosures of which are incorporated herein by reference in their entireties.

### BACKGROUND

### Field

The present disclosure relates to a device for collecting a quantity of perspiration excreted by a subject, in particular by the eccrine sweat glands of the subject, and, if appropriate, used for the measurement of at least one parameter of the perspiration.

### Brief Description of Related Developments

Measuring, e.g., the amount of perspiration excreted can be useful for athletes and more generally for all people who need to know the volume of water they lose during an episode of perspiration triggered by the thermoregulation system of the body. Such measurement is used for monitoring a person's level of hydration. Moreover, the analysis of the composition of perspiration can also be used for diagnosing certain diseases or abnormalities, e.g. by determining the concentration of different ions and organic compounds contained in perspiration.

The publication by Koh et al., entitled “A soft, wearable microfluidic device for the capture, storage, and colorimetric sensing of sweat” published on 23 Nov. 2016 in the 8^{th} volume of the journal “Science Translational Medicine” (366ra165 (2016)), describes a device for determining, by colorimetry, the concentration of glucose, lactate, chloride ions and hydronium ions (pH). Such device can also be used for determining the total amount of perspiration, the rate of sweating and the variations thereof.

Such device includes a flexible patch which integrates a microfluidic circuit, which comes out via separate channels into four chambers, each containing a reagent for a colorimetric determination of the concentration of glucose, lactate and chloride ions, and the level of pH, respectively. Such reagents are also known for said use. The microfluidic circuit is formed in a poly dimethyl siloxane (PDMS) layer. The microfluidic circuit is attached to the skin by a perforated adhesive layer that lets perspiration through. An RFID chip, isolated from the microfluidic circuit, equips the patch and is used for the communication with a mobile smartphone.

The above-mentioned device operates as follows: the patch is applied so that the adhesive layer is in contact with the skin of the patient. Excreted sweat enters the microfluidic circuit and fills the circuit. Sweat penetrates into the chambers containing the reagents: the color of sweat changes on contact with each of the reagents. The results are obtained using a mobile phone. The mere fact of approaching a mobile phone to the patch triggers the taking of images by the mobile phone, which determines the absorbance of each of the zones of the microfluidic circuit. Since the absorbance is correlated with the concentration of the solute reacting with the reagent, the solute concentration of sweat is conventionally deduced therefrom.

The measurement of the angle of the filling front of the microfluidic circuit (the front corresponds to the change of color) by the mobile phone is used for determining the volume of sweat collected, which is used for calculating the total quantity of sweat excreted, and, depending on the measurement time, the speed of sweating.

The publication “Adhesive RFID sensor patch for monitoring of sweat electrolytes” by Daniel P. Rose et al, published in June 2015, in volume 62, No. 6 of the journal IEEE Transactions on biomedical engineering, pages 1457-1465, describes a device used for measuring the amount of Na+ ions contained in the perspiration. The device includes a patch which comprises a porous adhesive coated with a cut-out sheet of polyamide. The perforated sheet includes an antenna which surrounds the hole and which consists of a thin layer of copper. In the hole, there is a laser-cut microfluidic paper which defines a collection compartment. The two terminals of the antenna are arranged at the collection compartment. A chip is placed above the antenna and covered with a gas-porous film.

The patch is attached to a part of the body of a patient. The sweat excreted by the patient crosses through the porous adhesive and spreads into the microfluidic circuit. When the perspiration fills the collection compartment, a mobile phone is brought close to the aforementioned antenna, which sends an electromagnetic wave which generates an induced current in the antenna. The measurement of the conductance of the perspiration contained in the compartment is used for deducing the concentration of sodium chloride in the perspiration of the subject.

The above-mentioned devices collect perspiration in liquid form. However, in practice, especially when the subject makes a physical effort, it turns out that perspiration is also in vapor form; the body being hot, liquid sweat quickly turns into vapor. The aforementioned patches thus tend to peel off and are difficult to use, either in hot weather or when the user makes a prolonged physical effort. Moreover, it is difficult to obtain a clear front because the microfluidic circuit contains vapor that condenses here and there on the walls, also making it difficult to measure the advance of the front.

In addition, it has been found that the surface area of perspiration has to be large enough so as to be able to collect the perspiration excreted by a plurality of eccrine sweat glands, preferentially at least ten, in order to minimize the risk of error. The amount of perspiration secreted then rapidly fills the microfluidic channels, which then have to be very long.

It also turns out that when the microfluidic channel is long, the perspiration exuded at the channel collection point or “inlet” has an increasing difficulty in pushing the perspiration contained in the channel as the channel fills up. As a result, the sweat gland can no longer easily excrete perspiration. The functioning thereof is altered: the sweat gland no longer secretes perspiration or in any case no longer with the same kinetics; the sweat gland can also be damaged and burst under the pressure of the perspiration same contains and can no longer excrete. Similarly, the size of the patch that collects sweat in a microfluidic channel is proportional to the size of the collection point and to the length of the channel used for storing the sweat (by spreading inside the microfluidic channel) and for measuring the amount thereby collected. Therefore, such devices have a non-breathable surface area of one square centimeter to several tens of square centimeters, which has to be attached on the body in a lasting way (at least one hour). Sticking a too large, non-breathable surface on the skin can also cause many problems. For example, problems related to sweat that accumulates under the patch and can possibly enter the collection hole and distort the measurement, or suffocate, potentially until bursting (depending on the type of adhesive and the duration of use) the functioning of the glands under the entire surface of the patch (making the next patches impossible to put back in the same place whereas in principle an individual should place the patch always in the same place, session after session). In order to limit such problems, the sweat collection points or inlet of the microfluidic channel are small, which limits the number of glands sampled and hence the accuracy of the measurement. However, even with a small collection point (between 0.03 cm² and 0.12 cm²), the length of the microfluidic channel remains large (between 40 cm and several meters). As a result, the patch still has a large non-breathable surface in contact with the skin.

### SUMMARY

The goal of the present disclosure is to propose a device which would overcome some or all of the aforementioned drawbacks associated with the devices of the prior art.

A particular goal of the present disclosure is to propose a device which for measuring the volume of sweat collected, by means of a spot or an areola which would be easily detectable and readable by means of measurement which can detect the contour and/or the colors of the latter.

Another goal of the present disclosure is to propose a device which can be used by a user performing a physical activity or more generally sweating by activating the thermoregulation system of the body (climate, intense work, work in protective clothing, hormonal diseases, night sweating).

Another goal of the disclosure is to propose a device for detecting the presence of lactates in the perspiration and which optionally can be used for a concentration measurement of the latter.

The present disclosure relates to a device for collecting a fraction of the perspiration excreted by a subject, said device being of the type comprising a flexible patch, which comprises a lower face, said device including means of attaching said patch against the skin of said subject.

In a characteristic way, according to the disclosure, said patch is a multilayer patch which includes:
a first layer, the lower face of which at least partially forms the lower face of the patch, said first layer including at least one perforation defining a perspiration-collecting aperture able to be brought into direct contact with the skin of the subject, and a transfer opening;
a hydrophilic absorbent layer arranged above said first layer and communicating with the exterior environment, said absorbent layer communicating with said first layer through said transfer opening;
a film which can be transparent, if appropriate, and impervious to water and vapor, which covers at least the upper surface of said absorbent layer, and, furthermore, means of regulating the flow-rate of liquid and/or vapor perspiration penetrating into said absorbent layer and means of releasing the air contained in said absorbent layer towards the outside of said device.

The Applicant has indeed observed that regulating the flow-rate of perspiration penetrating into the absorbent layer makes it possible not to perturb the sweat glands of the subject. The Applicant further found that in the event of intense physical effort, most perspiration is rapidly in vapor form. Moreover, the Applicant found that the flow-rate of perspiration penetrating into the absorbent layer should be regulated in order to prevent a too rapid saturation of the latter. The latter absorbs liquid perspiration by capillarity due to the open pores thereof. When the absorbent layer is saturated, same can no longer collect perspiration. It follows therefrom that the device cannot be used for physical activities that are too intense and/or too long. The means of regulating the flow-rate of perspiration penetrating into the absorbent layer make it possible to use an absorbent layer with a relatively small surface area and with small thickness even for intense or long physical activities because such means prevent the rapid saturation of the absorbent layer.

The means of releasing air of the absorbent layer allow the latter to absorb perspiration by capillarity, the pores emptying from the air contained therein, in order to become filled with perspiration.

The absorbent layer is preferentially a layer that does not swell when sweat is absorbed and does not change size.

A fraction of the excreted sweat is thereby collected, which fraction can be used e.g. for measuring the total volume of sweat secreted by the subject or the presence of an ion or compound in the perspiration, in particular by measuring a spot with a visible areola, and making possible an easy optical detection of the volume of perspiration.

According to a first aspect, said means of regulating the flow-rate include a microfluidic channel coming out at said perspiration-collecting aperture and at said transfer opening formed in said first layer.

The channel, once filled with liquid perspiration (i.e. approximately e.g. 15 minutes to 20 minutes after the beginning of the physical effort), prevents vapor perspiration from penetrating into the absorbent layer; the perspiration condenses in liquid form. A spot or areola is thereby obtained having a clear contour and the surface of which is then easy to determine.

The microfluidic channel is advantageously provided in the thickness of said first layer. The latter could e.g. consist of three stacked sublayers, the central layer including a cutout forming the vertical walls of the channel.

The perspiration-collecting aperture can comprise elastically deformable lips able to come into contact with the skin of the user so as to make a watertight and airtight connection between the skin and said device, at the end of said perspiration-collecting aperture.

The means of releasing the air contained in said absorbent layer towards the outside of said device can include at least one air-permeable layer which is in contact with said absorbent layer and/or a channel open to the outside of said device and which communicates with said absorbent layer.

The microfluidic channel can include a buffer zone for storing perspiration and said buffer zone can communicate with said transfer opening. The buffer zone is used for controlling the flow-rate of liquid perspiration. When located below the transfer opening, the contact between the perspiration contained in the buffer zone and the absorbent layer (when the latter is discretely arranged on the first layer) occurs at the upper surface of the volume of perspiration, which leads to a fine regulation according to the volume of the buffer zone.

Advantageously, the perspiration-collecting aperture is arranged at a distance from said transfer opening, in a substantially horizontal plane parallel to the stacked layers of said patch. The length of the microfluidic channel can be on the order of a centimeter even when the perspiration-collecting aperture has a sufficient surface area for surrounding at least a dozen sweat glands (up to more than fifty).

As an example, when the perspiration-collecting aperture has a surface area of 0.2 cm² to 1 cm², the channel can have e.g. a length equal to or comprised between 1.5 cm and 7 cm. The collection of perspiration takes place mainly in the absorbent layer.

The communication between the absorbent layer and the outside of the patch is not limited according to the disclosure. Such communication can be either direct or indirect.

According to a variant of the first aspect, said first layer further includes a vent channel which comes out laterally out of said patch and which connects a vent opening provided in said first layer, said vent opening communicating with said absorbent layer. The absorbent layer can be arranged on the first layer, in which case the vent opening opens out under the absorbent layer.

Advantageously, said vent channel forms at least one meander, which makes it possible to reduce the evaporation of perspiration in the absorbent layer. Indeed, because of the heat of the body, perspiration tends to evaporate; a too direct contact with the drier outside air increases the evaporation, which then hinders the measurements made on the fraction of perspiration collected in the device of the disclosure.

The microfluidic channel can contain a water-soluble colored agent or a water-soluble reagent able to react with the perspiration. It is then possible to color the absorbent layer without modifying same, in particular without impregnating the layer with chemical compounds able to react with the perspiration. Liquid perspiration flowing through the channel will dissolve the colored agent or reagent and carry over the agent into the absorbent layer, forming e.g. a spot. It is also possible to increase the number of parameters relating to the perspiration of the subject and which can be measured from the device of the disclosure or by the latter. It is thereby avoided to put all the reagents or chemical detection agents in the absorbent layer.

According to a second aspect of the means of regulating the flow-rate of liquid and/or vapor perspiration penetrating into the absorbent layer, and which can be combined, if appropriate, with the first aspect, said transfer opening is opposite or corresponds to said perspiration-collecting aperture and said means of regulating the flow-rate include at least one intermediate layer arranged between said first layer and said absorbent layer and which includes an intermediate opening, having a surface area smaller than the surface area of said perspiration-collecting aperture, said intermediate opening being opposite said aperture and said transfer opening and said first layer, having a vapor diffusion coefficient CDV1 greater than the water vapor diffusion coefficient CVD2 of said intermediate layer and said intermediate layer being permeable to air and allowing air to be released from said absorbent layer to the exterior environment.

The absorbent layer is placed in communication with the exterior environment via the intermediate layer which lets air through, in particular, and via the perspiration-collecting aperture which corresponds to or is opposite to the transfer opening.

The patch has the merit of making migrate vertically the liquid perspiration initially excreted, preventing that too much vapor perspiration enters the absorbent layer and then condenses thereto, rapidly saturating the latter or forming poorly readable spots, with a fuzzy contour.

According to one aspect, the intermediate layer includes only the intermediate opening to the exclusion of any other perforation or opening. Such aspect is suitable when the intermediate layer includes sublayers with variable permeability to vapor.

Advantageously, in the second aspect, the first layer extends around the absorbent layer and is conformed so as to let vapor through towards the exterior environment of said patch. The release of vapor prevents the saturation of the absorbent layer. When the first layer includes an adhesive lower face, the face acts as a means of attaching the device to the skin of the user.

Advantageously, the surface area of the intermediate opening is smaller than the surface area of the perspiration-collecting aperture or the surface area of the transfer opening, so as to reduce the flow-rate entering the absorbent layer.

The combination of the intermediate layer with the first layer is used for regulating the flow-rate of perspiration by removing a large portion of the vapor perspiration before condensing the latter further away from the body, under the absorbent layer.

According to a variant of said aspect of the means of regulating the flow-rate, said first layer includes secondary perforations opening out under said patch and said means of regulating the flow-rate also further includes at least one first sublayer made of watertight material, arranged between said first layer and said intermediate layer, said first sublayer including a perspiration drainage circuit which connects said secondary perforations with the exterior of said patch.

The drainage circuit is also a microfluidic circuit. The height of the channel advantageously varies from 50 μm to 100 μm and the width thereof varies e.g. from 100 μm to 400 μm or 600 μm. The drainage circuit is an open circuit.

The drainage circuit limits the amount of liquid perspiration penetrating into the absorbent layer. The shape and the surface area of the discharge circuit are not limited according to the disclosure. The circuit has to drain the liquid or vapor perspiration condensing in the circuit and coming from the first layer towards the edges of the first sublayer, which are in the open air and thereby allow the perspiration to evaporate into the air.

The intermediate layer can include at least two sublayers having different and decreasing vapor diffusion coefficients, the sublayer in contact with said first layer having a vapor diffusion coefficient greater than the vapor diffusion coefficient of the sublayer in contact with said absorbent layer. In such aspect, all the sublayers have an identical perforation or main opening and all the perforations are arranged one on top of the other.

A stack of such sublayers prevents the vapor from remaining in contact with the skin and moves away or detaches the patch from the skin, thereby hindering the collection of perspiration. The absorbent layer can react with the vapor. Nevertheless, if the vapor comes into contact with the absorbent layer, small spots form, without a distinct front; the small spots can hinder the measurements subsequently performed on the spot. It could be preferable to limit the contact between the absorbent layer and the vapor.

According to a variant of the means of regulating the flow-rate of liquid and/or vapor perspiration, the intermediate layer includes at least three sublayers, a first sublayer of watertight material and including a main perforation arranged above the transfer opening of said first layer, a second sublayer, also watertight, arranged on said first sublayer and which forms a perspiration storage circuit, said storage circuit being isolated from the outside of said patch and putting said main perforation made in said first sublayer in communication with the lower surface of a third sublayer, said third sublayer also being made of a watertight material at least on the lower surface thereof, said third sublayer including at least one main perforation which puts said water storage circuit in communication with the lower face of said absorbent layer.

In such aspect, the liquid perspiration is stored in a storage circuit and then brought into contact with the absorbent layer at predetermined zones defined by perforations provided in the third sublayer. The size and the number of the areolas/spots which will serve e.g. for determining the amount of sweat excreted are thereby defined.

Advantageously, the main perforation of said third sublayer is arranged above said main perforation of said first sublayer.

The storage circuit can be formed by cutting the sublayer, the bottom of the circuit being formed by the surface of the layer located under and the circuit being covered by the layer above. The above also applies to the drainage circuit. A closed circuit is a circuit that does not communicate directly with the exterior environment. The storage circuit can be either microfluidic or not microfluidic. Same can have a dimension on the order of a millimeter or more.

According to a variant, the second sublayer includes a main perforation with a surface area greater than the surface area of the main perforation of said first sublayer and than the surface area of said third sublayer. Such perforation also serves for storing liquid perspiration.

Whatever the aspect, said layers and, if appropriate, the sublayers of said patch are rigidly attached to one another and are more particularly bonded to one another.

Whatever the aspect, the layers and, if appropriate, the sublayers are chosen independently from one another from acrylate, polyester and acrylic sheets and from the stacks of at least two or three of said sheets.

Whatever the aspect, the absorbent layer can be an asymmetric microporous or nanoporous membrane. Advantageously, said absorbent layer includes open pores the cross-section of which varies according to the thickness of said layer. The cross-section of the pores can advantageously decrease according to the thickness of the layer. Thereby, if the end of the pores with the largest cross-section is arranged towards the first layer, it is possible to block large molecules such as lactates in the absorbent layer; water, which is a smaller molecule, migrating to the surface of the absorbent layer located under the transparent film.

The hydrophobic and impermeable film is transparent when the determination of a parameter is performed by measuring the size and/or the color of a spot. The term “porous membrane” refers to a flexible sheet that can be folded or curved and has open pores, letting occur the phenomenon of absorption by capillarity.

Advantageously, whatever the aspect, the absorbent layer contains a reagent able to react chemically with perspiration by changing color or by modifying the electrical resistance of said absorbent layer. The device can then be used for measuring, either optically or electrically, a parameter which can then be correlated with a characteristic of the excreted sweat which can be e.g. the volume, the pH or the composition thereof.

Thereby, the device can include electrodes arranged so that the electrical resistance of said porous layer can be measured.

Furthermore, the device can further include a flexible metal circuit forming an antenna and a writable chip such as a RFID or an NFC connected to said antenna, said antenna surrounds said absorbent layer or when the antenna is present, said first sublayer forming said drainage circuit, both ends of said antenna being electrically connected to opposite edges of said absorbent layer or of said drainage circuit.

Such antenna makes it possible to determine the conductance of the perspiration and hence concentration thereof of ionic species, in particular sodium.

Advantageously, said transparent film includes or lets appear optical information able to start a dedicated application when the film is detected by a reading device.

Advantageously, the device of the disclosure includes means of measurement of the surface and/or the color of the spot formed by perspiration on the surface of said absorbent layer, under said impervious film and said means of measurement makes it possible to correlate the surface of the spot and/or the change of color and/or of intensity of said colors of said spot with at least one item of information selected from: the volume of perspiration excreted by the subject, the concentration of at least one compound or ion selected from lactates, nitrates, sodium, potassium, calcium, magnesium, acetic acid, propionic acid, butyric acid and uric acid.

Such means of measurement can be integrated into a mobile telephone such as a smartphone.

Advantageously, said reading means further include a remote power supply relay for said chip at a given frequency and said optical information is also able to start, when read by said optical reader, the power supply to said chip by means of said power supply relay at said given frequency.

The means of attachment of the patch are not limited according to the disclosure. According to a particular aspect, the means of attachment are permeable to gas and in particular to vapor. Same allow perspiration to evaporate around the patch and prevents damage to the sweat glands;

The means of attachment can include an elastically deformable armband on which said patch is mounted, said armband including a passage for said microfluidic channel and said perspiration-collecting aperture being arranged on the face opposite the face on which the absorbent layer is located.

The means of attachment of said patch can further include a flexible sheet which is, if appropriate, adhesive and said sheet is attached under said first layer and sticks out around the latter, said sheet including a passage for said microfluidic channel and said perspiration-collecting aperture being arranged on the other side of said sheet with respect to said absorbent layer. The sheet is preferentially permeable to gas, in order to prevent detachment thereof due to perspiration. The sheet advantageously adhesive, can avoid the use of the armband at the same time as the sheet.

The present disclosure further relates to a method for determining the quantity of sweat excreted by a subject, according to which:
a flexible patch according to the present disclosure is affixed to the skin of the subject,
the subject is made to sweat until the appearance of a visible spot through said transparent film;
the surface of said exposed absorbent layer is photographed through said transparent film;
a calibrated image of said absorbent layer is produced;
said calibrated image is binarized so as to be divided into pixels;
the color gradient of each pixel in the RGB system is calculated so as to identify the spot formed by the diffusion of perspiration in the absorbent layer;
   the pixels contained in the zone defined by the perspiration diffusion front, are extracted;
   the colors of said zone are determined in the RGB system, the density thereof and the surface areas each color occupies;
   the surface area values, the color and density obtained are compared with the data contained in a database so as to determine the amount of sweat excreted and, if appropriate, the concentration of lactates when said absorbent layer contains at least one coloring agent able to change color when the pH of perspiration is changed due to lactates, the spot then comprising a plurality of colors.

### Definitions

The vapor diffusion coefficient corresponds to the diffusion coefficient of water vapor. Same is measured as follows: the opening of a container containing a known volume of water is hermetically sealed with the microporous membrane or layer for which the vapor diffusion coefficient is to be measured. The surface of the opening is porous. The glue used for gluing the membrane or layer to the container is watertight and vapor-tight. The assembly is placed at 37° C., for 24 hours, at atmospheric pressure. The level of water is then measured. The difference in volume observed corresponds to the volume of liquid water that passed through the membrane in vapor form, during the 24 hours. The coefficient is thus expressed in g·m⁻²·j⁻¹.

The term “vapor diffusion coefficient” thus refers to the water vapor diffusion coefficient measured at 37° C. and at atmospheric pressure, according to the aforementioned method.

The term “water” refers to liquid water.

The term vapor refers to water vapor.

The coloring agent is advantageously selected from food coloring agents, such as chlorophyll, chlorophyllines, norbixin and astaxanthin, crajirule extract, cresol, 3-methylphenol, 2-chloro-4-[3-(3-chloro-4-hydroxyphenyl)-1,1-dioxobenzo[c]oxathiol-3-yl]phenol, 2-chloro-4-[3-(3-chloro-4-hydroxyphenyl)-1,1-dioxobenzo[c]oxathiol-3-yl]phenol, 4,4′-(1,1-Dioxido-3H-2,1-benzoxathiole-3,3-diyl)bis(2-bromo-6-isopropyl-3-methylphenol), 3,3′-dibromosulfone gallein, le 3′,3″-dibromo-p-xylenolsulfonphthalein, bromophnol blue, phenolphtalein, 3,3-Bis(4-hydroxy-3-methylphenyl)-1(3H)-isobenzofuranonein, bromocresol green, bromothymol blue, copper phthalocyanine, and anthocyanins the derivatives thereof, more particularly betacyanin, curcumin, carotenoids including beta-carotene (trans form), flavonoids, more particularly catechin, quercetin, apigenin and luteolin.

Preferentially, a mixture of coloring agents is chosen, one coloring agent of which changes color at pH 7 and the other changes color at a pH less than 5.5. For example, a mixture of bromocresol green and bromothymol blue is used. Such mixture can be used for properly impregnating the hydrophilic microporous membrane and changes color at pH=7 and at a pH less than 5.5. The indicates, by the two changes of color thereof, the passage of water and the passage of lactates in the micro porous membrane (absorbent layer).

According to the present disclosure, a microfluidic channel is defined as being a channel having at least one dimension on the order of a micrometer. The channel can be advantageously provided in a hydrophobic material. The channel has a bottom, two, three or four vertical walls. The channel can have a ceiling; same then forms a cavity which can be open, if appropriate, to the outside by openings provided in the bottom, the ceiling and/or the vertical walls.

The absorbent layer is not limited according to the disclosure. Same can be a hydrophilic porous PVDF membrane, a polypropylene membrane made hydrophilic by grafting, e.g. a membrane made of polysulfone or of polyether sulfone.

Advantageously, the absorbent layer which has an asymmetric porosity is a polysulfone membrane or a polyether sulfone membrane. The Applicant has found that such membranes are easy to impregnate with water-soluble coloring agents and more particularly with bromocresol green and/or bromothymol blue. Such a membrane impregnated with the coloring agent mixture remains stable and does not change color until the patch is used. The coloring agents remain homogeneously distributed in the microporous membrane without altering the latter. Such a membrane does not swell and does not change size.

A person skilled in the art is able to determine, by routine experiments, the thicknesses of the different layers and sublayers as well as the porosity and the vapor diffusion coefficient thereof, in order to determine which combination is preferable in the case of perspiration.

Nevertheless, the Applicant has the merit of having shown that a perfectly bonded patch is obtained which leads to obtaining at least one readable spot formed by the contact of water with the colored membrane when the flow-rate of perspiration entering the absorbent layer is regulated. The first layer in contact with the skin advantageously has a thickness substantially equal to or greater than 50 μm and less than or equal to 120 μm (including the adhesive layer). The intermediate layer formed, if appropriate, of the sublayers, has a thickness substantially equal to or greater than 100 μm and substantially equal to or less than 300 μm (thickness of the adhesive layer bonded, if appropriate, to the layer or sublayer comprised). The values of the aforementioned thicknesses are advantageously coupled to the following vapor diffusion coefficients: the lower layer has a vapor diffusion coefficient greater than 3000 g/m²/d. The intermediate layer has a vapor diffusion coefficient substantially equal to or greater than 300 g/m²/d and substantially equal to or less than 1200 g/m²/d whether the layer is single layer or formed of a plurality of sublayers. The aforementioned thicknesses and vapor diffusion coefficients make it possible to obtain a thin and flexible patch that follows well the movements of the body of the user and does not detach.

The first layer has a vapor diffusion coefficient on the order of 2, 2.5 or 3 times the greatest vapor diffusion coefficient of the sublayers forming the intermediate layer.

The subject is not limited according to the disclosure. The subject can be a human or an animal.

### BRIEF DESCRIPTION OF THE FIGURES

The present disclosure, the features thereof and the advantages the disclosure brings abouts will become more apparent upon reading the following description of three aspects and of one variant, presented as examples, but not limited to, and which refer to the enclosed drawings wherein:

FIG. 1 is a perspective exploded view of a first aspect of the disclosure;

FIG. 2 is a top view of the device shown in FIG. 1;

FIG. 3 represents a perspective and exploded view of a second aspect of the patch of the disclosure;

FIG. 4 represents a perspective exploded view of a third aspect of the patch of the disclosure;

FIG. 5 represents a top view of the last two layers arranged before the absorbent layer of a variant of aspect of the third aspect;

FIG. 6 represents a photograph of the spot formed on the upper face of the microporous membrane (absorbent layer) by the perspiration, the spot being analyzed by the smartphone equipped with a camera;

FIG. 7 represents a microporous membrane (absorbent layer) equipped with an antenna used for determining the conductance of the perspiration.

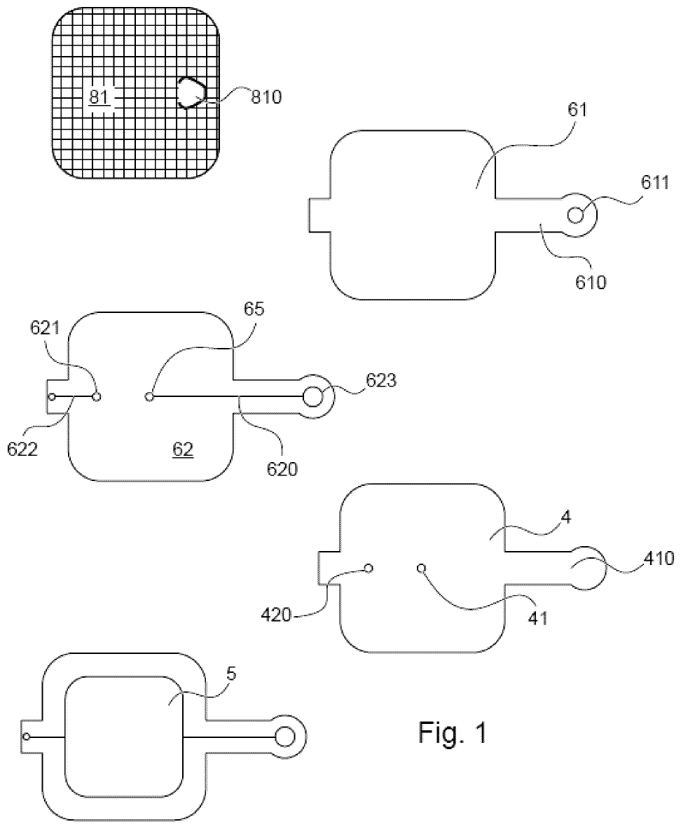

With reference to FIG. 1, the device of the disclosure includes a flexible patch 1. The patch is formed by a stack of layers rigidly attached to each other but not all having the same shape. In FIG. 1, the layers are shown from the layer closest to the skin of the subject, to the layer furthest from the subject when the patch is applied to the skin of the subject. In such aspect, the device includes a gas-permeable attachment sheet 81 which has an adhesive lower face which will be adhesively applied to the skin of the subject. The attachment sheet 81 includes a passage 810 forming a hole or a flap letting through the microfluidic channel, as is subsequently explained.

### DETAILED DESCRIPTION

With reference to FIG. 1, the microfluidic channel 6 is formed of three sublayers made of hydrophobic materials. The bottom of the microfluidic channel is formed by a sublayer 61 which includes a tab 610 ending in a perspiration-collecting opening or aperture 611 surrounded by an elastically deformable lip (not shown). The walls of the channel are formed by a second sublayer 62 which includes a micro-cutout 620, which is rectilinear in the aspect shown. The rectilinear cutout 620 opens out into a cutout 623 of the same shape and arranged opposite the aperture 611 and in a buffer zone 65. The second sublayer 62 further includes a second channel 622, distinct from the first channel 610. This second channel 622 serves as a vent for the absorbent layer 5. The second channel 622 opens out to the outside of the second layer 62 and starts at an aeration perforation 621. The sublayer 62 forms the vertical walls of the microfluidic channel and thus the thickness of the latter.

The third sublayer closing the microfluidic channel is the barrier layer 4 which is hydrophobic and impermeable to gases and liquids. The barrier layer 4 includes a transfer opening 41 which is placed above the buffer zone 65 and a vent opening 420 arranged opposite the ventilation perforation 621 of the second sublayer 62. The barrier layer includes a tab 410 which closes the top of the cutout 620 and the top of the cutout 623.

Thereby, the stacked layers 61, 62 and 4 form the first layer of the patch. All the sublayers 61, 62 and 4 are made of hydrophobic and liquid-tight materials, in particular watertight.

The absorbent layer 5 is arranged over the barrier layer 4. A sealed and transparent film 7 covers the absorbent layer 5 and extends over the layer 4, covering the tab 410.

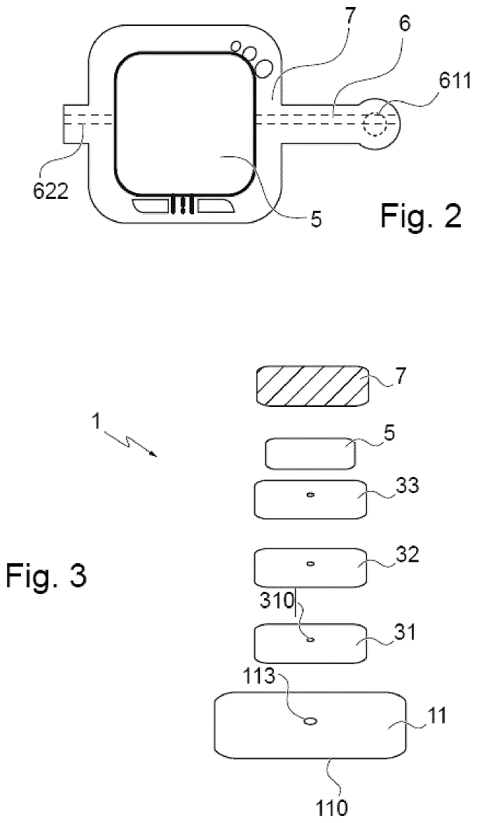

FIG. 2 is a top view of the patch 1. The channels 6 and 622 are shown in dotted lines since same are located in the thickness of the patch 1 or under the latter. It can be seen in FIG. 2 that the perspiration-collecting aperture 611 is far from the absorbent layer 5 and that the latter communicates with the outside of the patch 1 via the vent channel 622.

It will be described, with reference to FIGS. 1 and 2, how such device works. The tab including a part of the microfluidic channel 6 is passed through the opening 810 of the attachment sheet 81. The protection sheet is removed from the adhesive face of the attachment sheet 81 and the patch is adhesively applied to the skin of the user by means of the attachment sheet 81. The perspiration-collecting aperture 611 is thus applied to the skin of the user. The deformable lip that the aperture has on the circumference thereof guides the perspiration into the channel 6. The patch is located on the attachment sheet 81. Same can be bonded thereto.

When the user perspires, the perspiration collected at the perspiration-collecting aperture 611 enters the microfluidic channel 6. Due to the hydrophobic material forming the channel and to the size of the channel, the perspiration flows towards the buffer zone 65 and accumulates thereto. The perspiration in the form of vapor penetrates into the layer 5. When the entire buffer zone is filled with liquid perspiration, the surface of the liquid comes into contact with the absorbent layer 5 at the transfer opening 410. Liquid perspiration is absorbed by capillarity in the absorbent layer. Channel 6 continues to fill up due to the perspiration of the user. The absorbent layer 5 prevents the blocking of sweating by always absorbing a quantity of liquid overflowing from the buffer zone. The air initially contained in the channel 6 and in the absorbent layer 5 is released via the vent channel 622, which allows the absorbent layer to always absorb liquid perspiration.

Liquid perspiration penetrates into the absorbent layer 5 and reacts chemically with the reactants contained therein or modifies the conductivity thereof. If the reagent is a colored reagent, a colored spot appears slowly on the surface of the layer 5 located under the film 7. The size of the spot is proportional to the amount of liquid perspiration collected by the absorbent layer. The color of the spot can vary depending on the pH of the perspiration or on the presence e.g. of lactates.

To detect the modification of the parameter (herein, the color of the absorbent layer visible through the transparent film 7), reference will be made to the way the second aspect works, as indicated hereinbelow.

A second aspect will now be described with reference to FIG. 2 and following. With reference to FIG. 3, the device of the disclosure includes a flexible patch 1. The patch 1 is flexible and has a lower face which can be adhesively applied to the skin of the subject and follows the movements of the latter without detaching. The patch can be adhesively applied, e.g. to an arm or a forearm. The patch is made of material(s) suitable for being in contact with the skin.

The device of the disclosure further includes (like the first aspect) a mobile phone such as smartphone (not shown) equipped with a camera which acts as an RGB colorimeter.

With reference to FIG. 3, according to a first aspect, the patch 1 includes a first layer 11 or peel layer, the lower face 110 of which can be adhesively applied to the skin of the user. The peel layer is also used for attaching the patch to the skin. The peel layer 11 is made of a material able to come into contact with the skin of a subject, in particular a human. The same applies to the glue which covers the lower face 110 thereof. The peel layer 11 has a central perforation or main perforation 113 the surface area of which is comprised between 3.14 mm² and 12 mm². The peel layer 11 is permeable to water and vapor. The patch 1 further includes a microporous membrane 5 (absorbent layer) which includes at least one coloring agent or a mixture of water-soluble coloring agents and which changes/change color according to the pH. The microporous membrane 5 is covered with a transparent film 7 impervious to water and vapor. The patch 1 further includes an intermediate layer which is formed, in the present case, of a stack of three sublayers 31, 32 and 33. The three sublayers 31, 32 and 33 are perforated at the center thereof and the perforations thereof are arranged opposite each other and opposite the perforation 113 of the peel layer 11. The perforations of the three sublayers 31, 32 and 33 are identical and have a surface area smaller than the surface area of the perforation 113 of the peel layer 11. In FIG. 1, in order not to clutter the figure, only the main perforation 310 of the sublayer 31 is referenced.

In such particular aspect, the sublayers 31, 32 and 33 are smaller than the peel layer 11, which means that the latter sticks out beyond the sublayers 31, 32 and 33, all around the sublayers. The film 7 seals the microporous membrane 5 and the intermediate layer on the peel layer 11 but does not cover the entire surface of the latter. The area of the peel layer 11 which is not covered by the film 7 nor by the intermediate layer allows the vapor to cross the peel layer and evaporate from the patch towards the ambient air. The three sublayers 31, 32 and 33 have decreasing vapor diffusion coefficients. The layer 31 thus has a vapor diffusion coefficient CDV1 greater than the vapor diffusion coefficient CDV2 of the sublayer 32. The sublayer 33 has a vapor diffusion coefficient CDV3 less than CDV2.

The three sublayers 31, 32 and 33 can be formed in porous membranes which are reinforced, if appropriate, by fibers, in particular polyolefin fibers, or e.g. in woven, knitted or non-woven textiles. Such textiles and membranes can be made of acrylate(s), acrylic, polyester(s), polyurethane.

A person skilled in the art is able to determine the vapor diffusion coefficient of each layer or sublayer experimentally.

As an example, but not limited to, whatever the aspect, the peel layer can be cut from a polyurethane strip, one face of which is glued with acrylate glue suitable for contact with the skin.

Similarly, whatever the aspect, the sublayer 31 can be a layer of adhesive such as perforated acrylate or a nonwoven fabric, e.g. of polyester, one face of which is covered with acrylate adhesive suitable for a contact with the skin.

Whatever the aspect, the sublayers 32 and 33 can be layers of acrylate adhesive reinforced by polyolefin fibers.

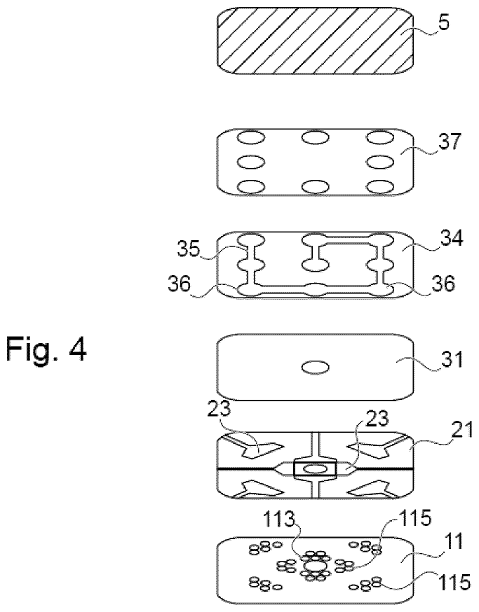

It will now be explained how such aspect works, with reference to FIG. 3. The patch 1 is adhesively applied on the skin of the user. During an effort or under the effect of heat, the user perspires. Perspiration is excreted in the form of liquid but also in the form of vapor. Throughout the application, perspiration is assimilated to water. The liquid perspiration will cross through the peel layer 11, at the perforation 113 thereof but also across the entire surface thereof because the peel layer 11 is permeable to water and vapor. The vapor evaporates out of the device over the entire area of the peel layer 11 which is not covered by the film 7 and/or the intermediate layer. Under the intermediate layer, the liquid and vapor perspiration passes through the perforation 113 and the vapor, to a lesser extent, passes through the sublayers 31, 32 and 33 of the intermediate layer. Since the sublayers 31 to 33 have a decreasing vapor diffusion coefficient, said sublayers act as a vapor barrier and only liquid perspiration passes through the central perforations of the sublayers 32 and 33. When the liquid perspiration fills the perforation of the sublayer 33, the perspiration comes into contact with the microporous membrane 5. The perspiration penetrates into the latter and eluates the coloring agent or the mixture of water-soluble coloring agents contained in the microporous membrane 5. The mixture of perspiration-coloring agent continues to migrate into the thickness of the microporous membrane 5 and forms an areola/spot on the upper surface of the latter, which is located under the transparent film 7. The areola or spot formed and the method for determining the amount of perspiration will be described more extensively with reference to FIG. 4.

With reference to FIG. 4, a second aspect of the patch of the device of the disclosure will now be described. The elements in common with the first aspect are referenced identically. In FIG. 4, the film 7 is not shown. In the second aspect, the peel layer 11 includes a central perforation 113 and 8 other groups 115 of perforations surrounding the central perforation 113 and distant from the latter towards the free edge of the peel layer 11. The peel layer 11 is surmounted by a first watertight sublayer 21. A microfluidic discharge circuit 23 is provided in said sublayer. The sublayer 21 is perforated in order to form the discharge circuit 23 which is in two parts. A part of the discharge circuit 23 opens out above the perforations 115 provided in the peel layer 11 around the central perforation 113. The other part opens out above the other groups of perforations 115 situated close to the free edge of the sublayer 21. The two parts of the circuit do not communicate with each other. Each part of the channels which connect the zones of the circuit 23 situated above the perforations 115 of the peel layer 11 to the free edge of the sublayer 21. The circuit 23 is thereby an open circuit which conducts the liquid/vapor perspiration which passes through the peel layer 11 to the outside of the patch 1.

With reference to FIG. 4, the intermediate layer includes a sublayer 31 made of watertight material. The sublayer 31 is perforated at the center thereof. The sublayer 34 which covers the sublayer 31 is made of a watertight material. The sublayer 34 includes a microfluidic storage circuit 35. The microfluidic storage circuit 35 is closed. Same emerges at the central perforation of the sublayer 31 and then extends along the perimeter of the sublayer 31 in a channel opening into storage cavities 36. The sublayer 34 is covered with a third sublayer 37 which is watertight at least on the lower face thereof (i.e. the face adhering to the sublayer 34). The third sublayer 34 has 8 perforations which are arranged opposite the storage cavities 36 of the second sublayer 34. The upper face of the sublayer 34 comes into contact with the membrane 5, which is covered by the transparent film 7 (not shown). The peel layer 11 and the other sublayers 31 to 37 have the same surface area and are stacked and bonded to one another.

It will be explained how the second aspect works, with reference to FIG. 4. Like in the first aspect, the patch 1 is applied to the skin of the subject. The perspiration in liquid and vapor phase crosses through the peel layer 11. The perspiration accumulates in the liquid state in the discharge circuit 23 of the first sublayer 21 and then leaves via the channels opening out at the free edge of the sublayer 21. Only at the central perforation 113, the water migrates through the perforation of the sublayer 31 and reaches the storage circuit 35. The storage circuit 35 is filled with perspiration as perspiration is produced. At each storage cavity 36, the water passes progressively through the last sublayer 37. As soon as a storage cavity is filled, the water from said cavity passes through the last layer and penetrates into the microporous membrane 5. An areola is then formed at the filled storage cavity 36. The number of areolas and the size thereof indirectly indicate the amount of perspiration excreted.

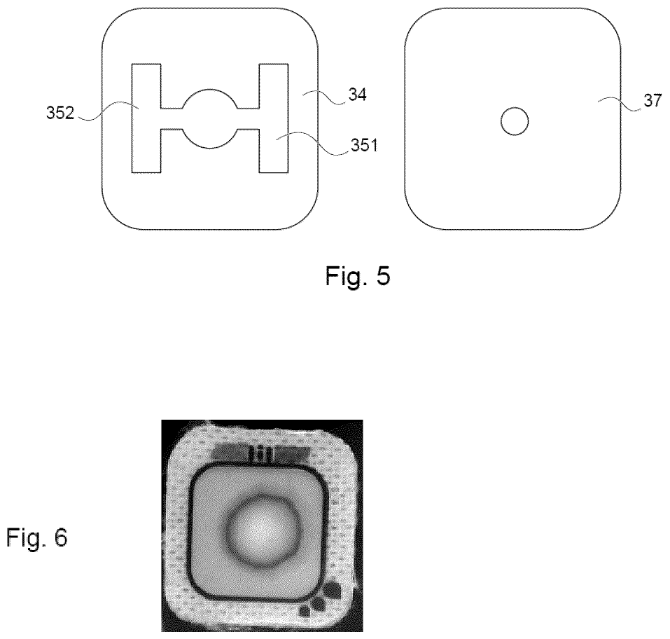

A third aspect will now be described with reference to FIG. 5. Such aspect is a variant of the second aspect. In such aspect, only the sublayers 34 and 37 are different. In FIG. 5, only the sublayers 34 and 37 are shown. The sublayer 34 has a larger central perforation which is also used for water storage. The perforation communicates via two channels with two lateral storage zones 351 and 352 which store water. The storage circuit 35 thus has a different shape from the aspect shown in FIG. 4. The last sublayer 37 has only one central perforation with the same surface area as the surface area of the sublayer 31.

Such variant works in the same way described with reference to the second aspect. The only difference is that only an areola is formed at the microporous membrane, opposite the central perforation of the peel layer 11 and of the sublayers 31, 34 and 37, which all communicate with each other vertically, like in all aspects of the disclosure. The size and/or the color of the areola can be used for determining the amount of perspiration excreted.

With reference to FIG. 6, it can be seen that the areola has a zone close to the perforation of the last sublayer which has a different color from the color of the microporous membrane. Such zone of different color is surrounded by a darker areola of the same color as the coloring agent or coloring agent mixture comprised in the microporous membrane. The shade is darker. The dark areola corresponds to the elution front of the coloring agent without a change in pH. Such zone contains more coloring agent than the rest of the surface of the membrane. The central zone has changed color due to the pH of the perspiration which is different from the pH of the microporous membrane. Moreover, in this specific case, since the microporous membrane is asymmetric, small molecules like water will quickly pass through it. Larger molecules, such as lactates that are contained in the perspiration, will be retained in the membrane and change the pH of the membrane. The coloring agent which is not eluted by water and is still present in the membrane will change color. The change in color is thus an indication of the presence of lactates in the perspiration. The coloring agent or coloring agent mixture is skillfully selected in order to change color upon a change of pH due to a retention of lactates.

The pictograms in the form of a black drop that decorate the black frame surrounding the membrane 5 covered by the film 7 are optical information leading to starting the dedicated application when taking a photograph of the patch of the disclosure.

The method for determining the amount of perspiration by analyzing the image of the areola or the areolas formed will now be described.

The user takes his/her mobile phone and photographs the transparent film 7 through which the areola is visible. Taking an image of the logo or of another graphic or optical sign identifies the patch 1 and starts a dedicated application. The patch 1 is identified, like with an object recognition or face recognition algorithm. Once the patch or patch 1 is identified, a plurality of photos are taken in order to form a sample. On the basis of the photos, a number of analyses of detection of reflections, geometric transformations and resizing are performed in order to correct the problems of angles of taking pictures and of orientation. A standardization/calibration of the dimensions and position of the different elements of the patch 1 is carried out in order to be able to determine the zone of analysis (the zone containing the areola) of the other zones of the patch 1.

Once the analysis zone is determined, the image of said zone is filtered and binarized. The gradient of each pixel in said zone is calculated using all the pixels in the near vicinity thereof. As a result, it possible to give a set of values corresponding to a direction and an identical intensity of color. Such method is used for identifying the different fronts formed by the liquid in the membrane and which correspond to the different colored zones of the areola.

Once the edges of the edges have been identified, the pixels contained in the zone described by the demarcation line of the front are extracted. The different surfaces of such zones are calculated, then the different colors are segmented by k-average clustering methods. The color zones and the density thereof are precisely obtained. The values obtained are compared with experimental values and the following are deduced therefrom: the quantity of perspiration excreted and the concentration of lactates by analysis of the relative surface occupied by the zone discolored by the lactates.

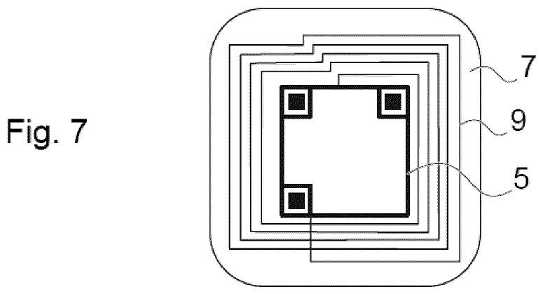

According to another variant of aspect shown in FIG. 7, the patch 1 includes an antenna 9 placed under the film 7 and which connects two opposite sides of the microporous membrane 5. The antenna 9 serves for measuring the conductance of the sweat soaking the microporous membrane 5. Conductance is used for determining the electrolyte concentration of the sweat. The antenna 9 is formed of a thin metal circuit; the antenna is flexible just like the rest of the patch and thereby provides good adhesion of the patch to the skin of the user. An RFID or NFC chip (not shown) is connected to the antenna 9 and makes it possible to record the conductance values of the reactive zone according to the signal sent by the mobile phone to the antenna 9. The antenna can be arranged at the other layers of the patch provided that the layer or the sublayer is indeed made conductive due to the presence of perspiration on the surface thereof, in the thickness thereof or in the microfluidic circuit the antenna includes.

The present disclosure further relates to all patches that are combinations of microfluidic circuits arranged inside or under the first layer and/or of the drainage circuit and/or storage circuit of liquid perspiration, arranged under the absorbent layer.

What is claimed is:

1. A device for collecting a fraction of the perspiration excreted by a subject, said device being of the type comprising a flexible patch, which comprises a lower face and means of attachment; of said patch on the skin of said subject, characterized in that said patch is a multilayer patch and in that the patch includes:
a first layer, the lower face of which forms at least partially the lower face of said patch, said first layer including at least one perforation defining a perspiration-collecting aperture able to be brought into direct contact with the skin of the subject, and a transfer opening;
a hydrophilic absorbent layer arranged above said first layer and communicating with the exterior environment, said absorbent layer communicating with said first layer through said transfer opening;
a film, which can be transparent, if appropriate, and impervious to water and vapor, covering at least the upper face of said absorbent layer;
and in that said device further includes means of regulating the flow-rate of liquid and/or vapor perspiration penetrating into said absorbent layer and means of releasing the air contained in said absorbent layer to the outside of said device.

2. The device according to claim 1, characterized in that said means of regulating the flow-rate includes:
a microfluidic channel coming out at said perspiration-collecting aperture and at said transfer opening provided in said first layer.

3. The device according to claim 1, characterized in that said means of releasing air contained in said absorbent layer towards the outside of said device include at least one air-permeable layer, which is in contact with said absorbent layer and/or a channel open to the outside of said device and which communicates with said absorbent layer.

4. The device according to claim 2, characterized in that said microfluidic channel is provided in the thickness of said first layer.

5. The device according to claim 2, characterized in that said microfluidic channel has a buffer zone for storing liquid perspiration and in that said buffer zone communicates with said transfer opening.

6. The device according to claim 1, characterized in that said collecting aperture is arranged at a distance from said transfer opening, in a plane substantially horizontal and parallel to the stacked layers of said patch.

7. The device according to claim 1, characterized in that said means of releasing air include a vent channel which emerges laterally outside of said patch and which connects a vent opening provided in said first layer and which communicates with said absorbent layer.

8. The device according to claim 1, characterized in that said transfer opening is opposite or corresponds to said perspiration-collecting aperture and in that said means of regulating the flow-rate include at least one intermediate layer arranged between said first layer and said absorbent layer and which includes an intermediate opening, having a surface area smaller than the surface area of said perspiration-collecting aperture, said intermediate opening being opposite said collecting aperture, in that said first layer, has a vapor diffusion coefficient greater than the water vapor diffusion coefficient of said intermediate layer and in that said intermediate layer is permeable to air and allows air to be released from said absorbent layer to the exterior environment.

9. The device according to claim 8, characterized in that said first layer has secondary perforations opening out under said patch and in that said means of regulating the flow-rate further include at least a first sublayer made of watertight material, arranged between said first layer and said intermediate layer, in that said first sublayer includes a drainage circuit for perspiration, which connects said secondary perforations with the outside of said patch.

10. The device according to claim 8, characterized in that said intermediate layer includes at least three sublayers, a first sublayer made of watertight material and having a main perforation arranged above the transfer opening of said first layer, a second sublayer which is also watertight, arranged on said first sublayer and forming a storage circuit for perspiration, in that said storage circuit is isolated from the outside of said patch and brings said main perforation provided in said first sublayer in communication with the lower surface of a third sublayer, in that said third sublayer is also made of a watertight material at least on the lower face thereof, in that said third sublayer has at least one main perforation which puts said storage circuit in communication with the lower face of said absorbent layer.

11. The device according to claim 1, characterized in that said absorbent layer contains a reagent able to react chemically with perspiration by changing the color or by changing the electrical resistance of said absorbent layer.

12. The device according to claim 1, characterized in that the device further includes a flexible metal circuit forming an antenna and a writable chip such as an RFID or an NFC connected to said antenna, in that said antenna surrounds said absorbent layer or, when present, said first sublayer form said drainage circuit, in that both ends of said antenna are electrically connected to opposite edges of said absorbent layer or of said drainage circuit.

13. The device according to claim 1, characterized in that said absorbent layer has open pores the cross-section of which varies according to the thickness of said layer.

14. The device according to claim 1, characterized in that the device includes means of measurement of the surface and/or the color of the spot formed by perspiration on the surface of said absorbent layer, under said impervious film and in that said means of measurement makes it possible to correlate the surface of the spot and/or the change of color and/or of intensity of said colors of said spot with at least one item of information selected from: the volume of perspiration excreted by the subject, the concentration of at least one compound or ion selected from lactates, nitrates, sodium, potassium, calcium, magnesium, acetic acid, propionic acid, butyric acid and uric acid.

15. A method for determining the amount of sweat excreted by a subject, wherein:
a flexible patch according to claim 1, is affixed to the skin of the subject,
the subject is made to perspire until the appearance of a visible spot through said transparent film;
the surface of said exposed absorbent layer is photographed through said transparent film;
a calibrated image of said absorbent layer is produced;
said calibrated image is binarized so as to be divided into pixels;
the color gradient of each pixel in the RGB system is calculated so as to identify the spot formed by the diffusion of perspiration in the absorbent layer;
the pixels contained in the zone defined by the perspiration diffusion front, are extracted;
the colors of said zone are determined in the RGB system, the density thereof and the surface areas each color occupies;
the surface area values, the color and density obtained are compared with the data contained in a database so as to determine the amount of sweat excreted and, if appropriate, the concentration of lactates when said absorbent layer contains at least one coloring agent able to change color when the pH of the perspiration is changed due to lactates, the spot then comprising a plurality of colors.
